(19) United States
(12) Patent Application Publication
(10) Pub. No.: US 2010/0312185 A1
(43) Pub. Date: Dec. 9, 2010

(54) DEVICE SUITABLE FOR ADMINISTERING MUTUALLY INCOMPATIBLE LIQUID SUBSTANCES INTO THE HUMAN BODY

(76) Inventors: Dario Petrone, Pignola (IT); Mirco Cavicchioli, Modena (IT)

Correspondence Address: Gregory A. Nelson;Novak Druce & Quigg LLP, 525 Okeechobee Blvd, Suite 1500, West Palm Beach, FL, 33401, US

(21) Appl. No.: 12/676,812

(22) PCT Filed: Sep. 8, 2008
(86) PCT No.: PCT/IT08/00571

§ 371 Date: Jul. 20, 2010

(51) Int. Cl. A61M 39/10; A61M 5/38

(52) U.S. Cl. 604/126; 604/533

(57) ABSTRACT

An object of the present invention is an apparatus for administering liquids into the human body characterized in that it has at least one main loading channel provided with at least one main administration point able to be connected to at least one container holding the liquid and at least one secondary loading channel, separated from the first by a membrane, provided with at least one container holding the liquid. Such apparatus allows the independent and simultaneous administration of liquids incompatible with each other, into the human body.

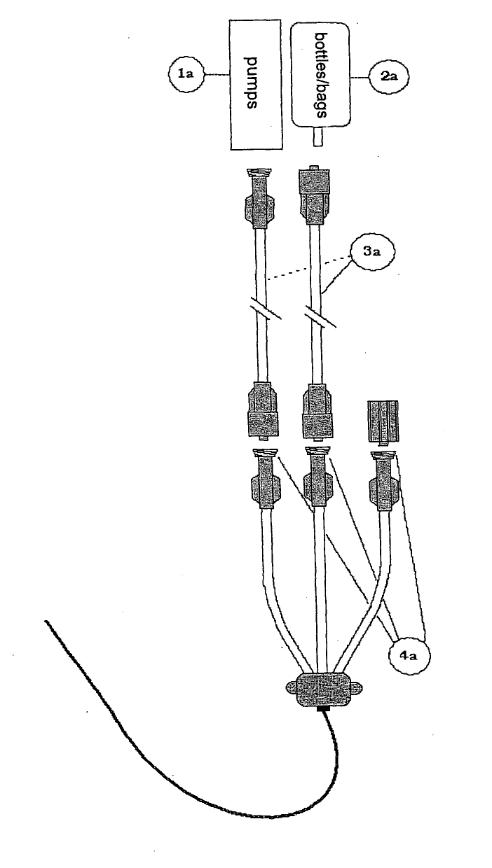

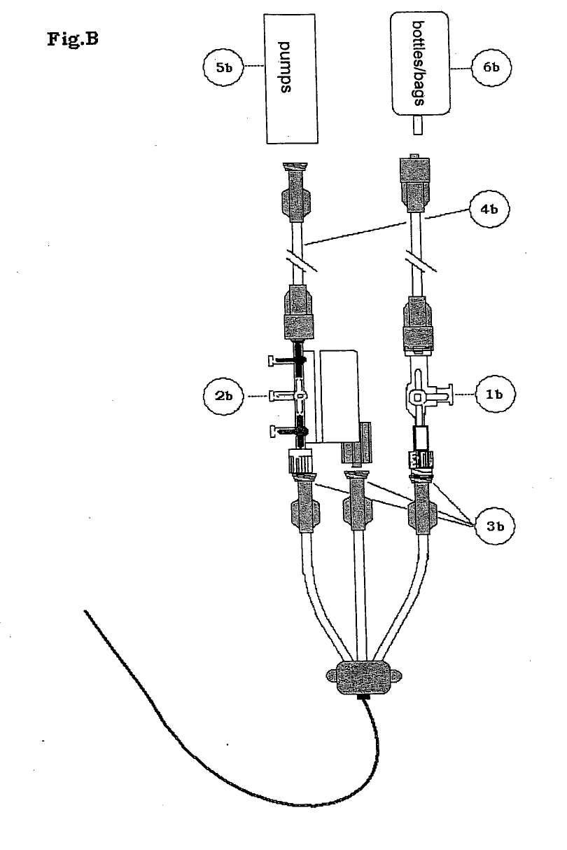

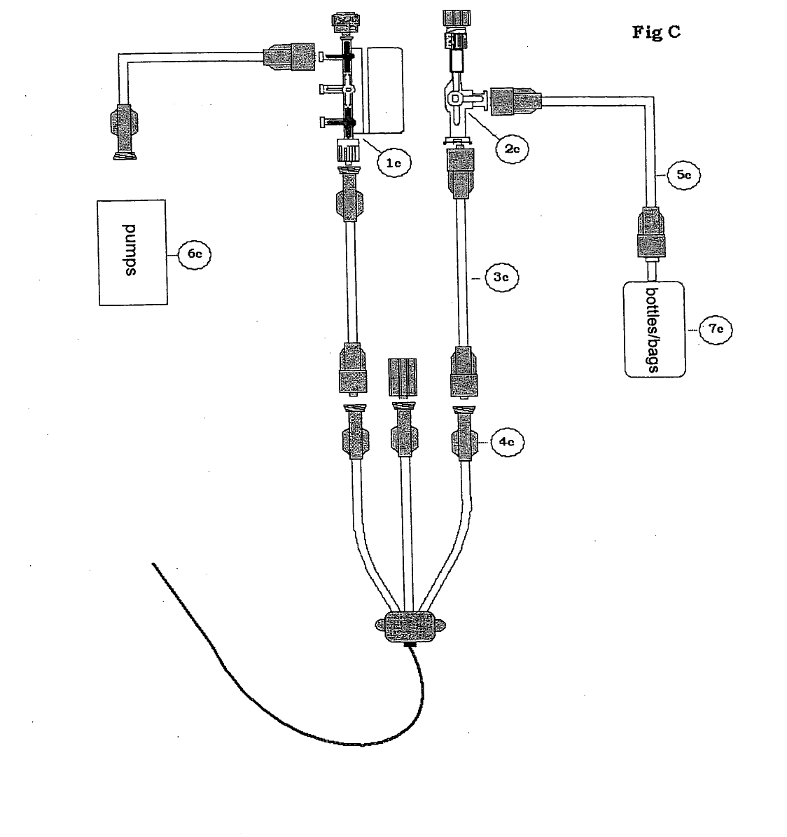

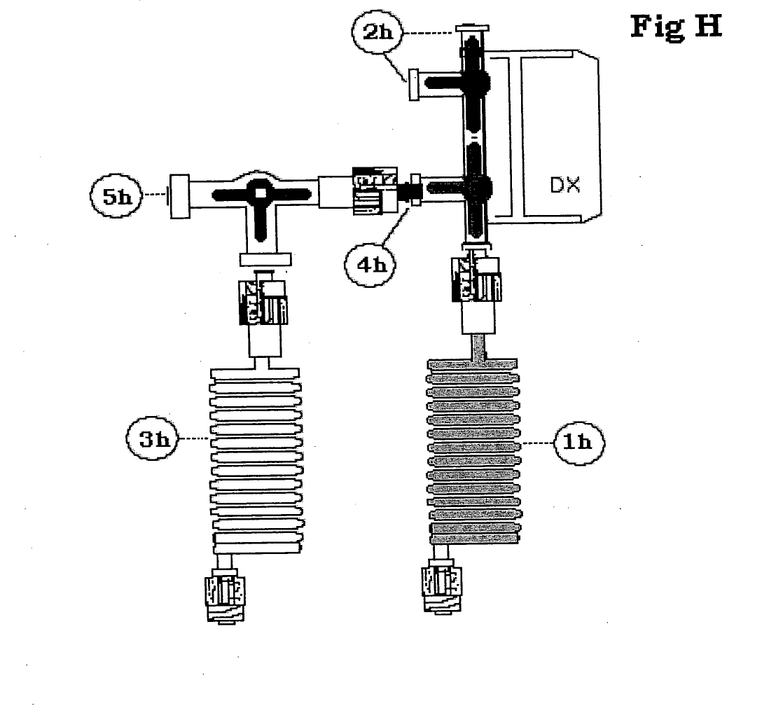

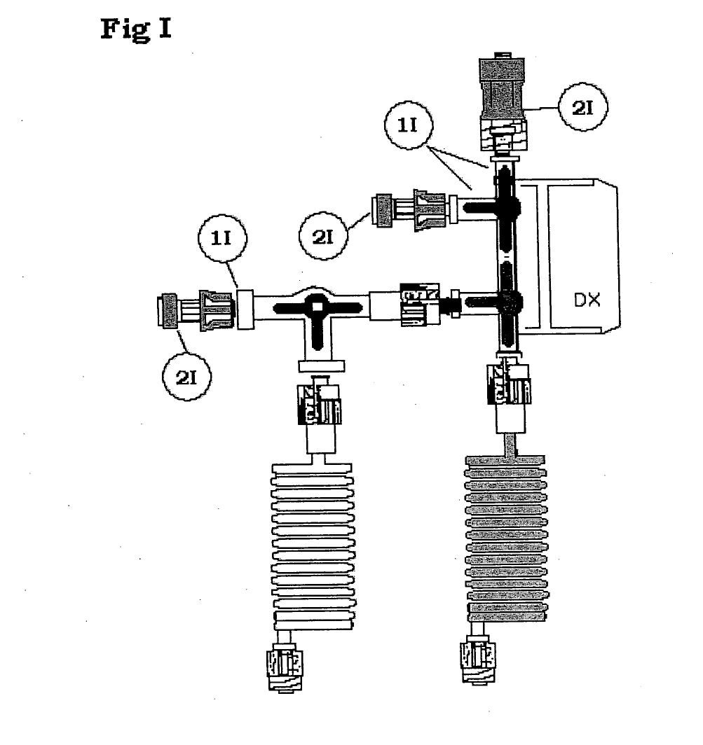

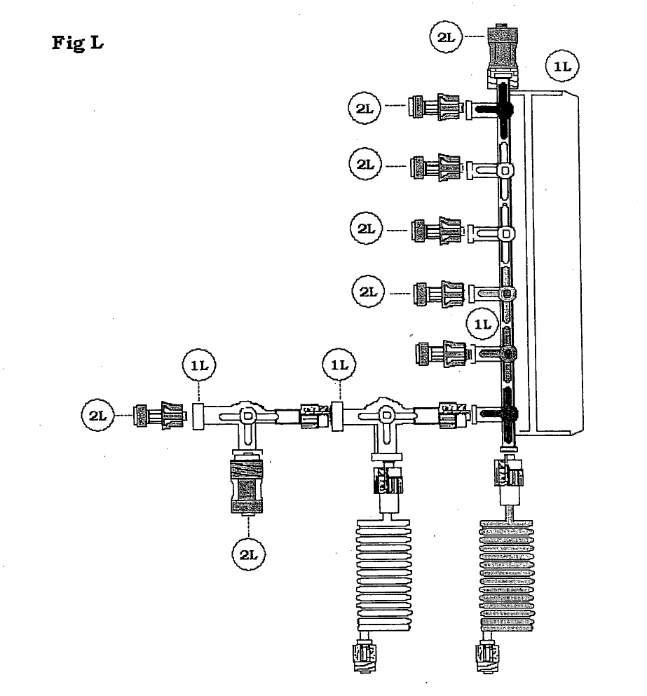

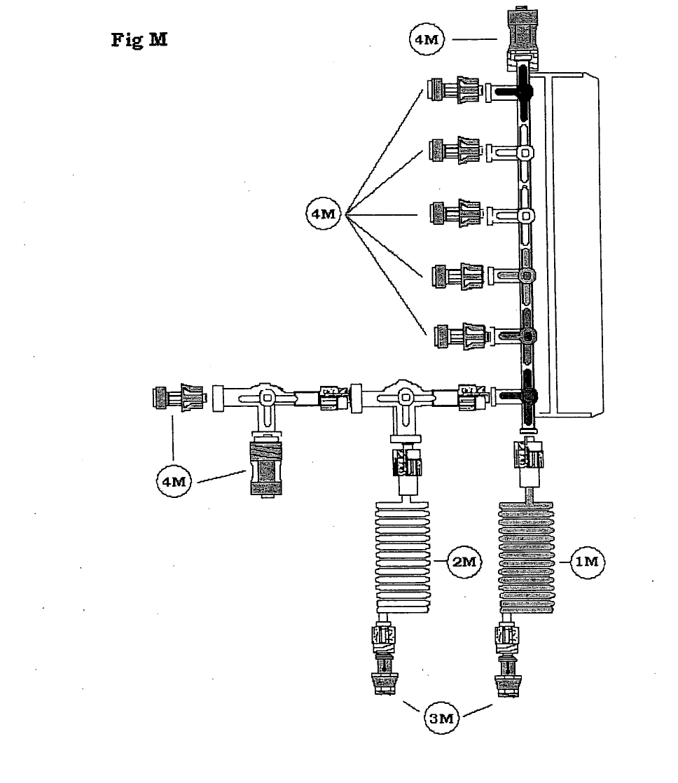

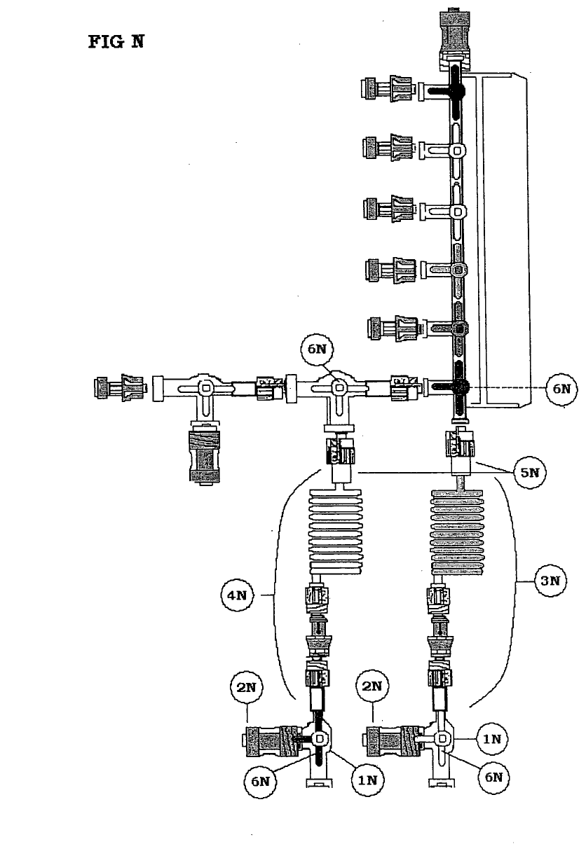

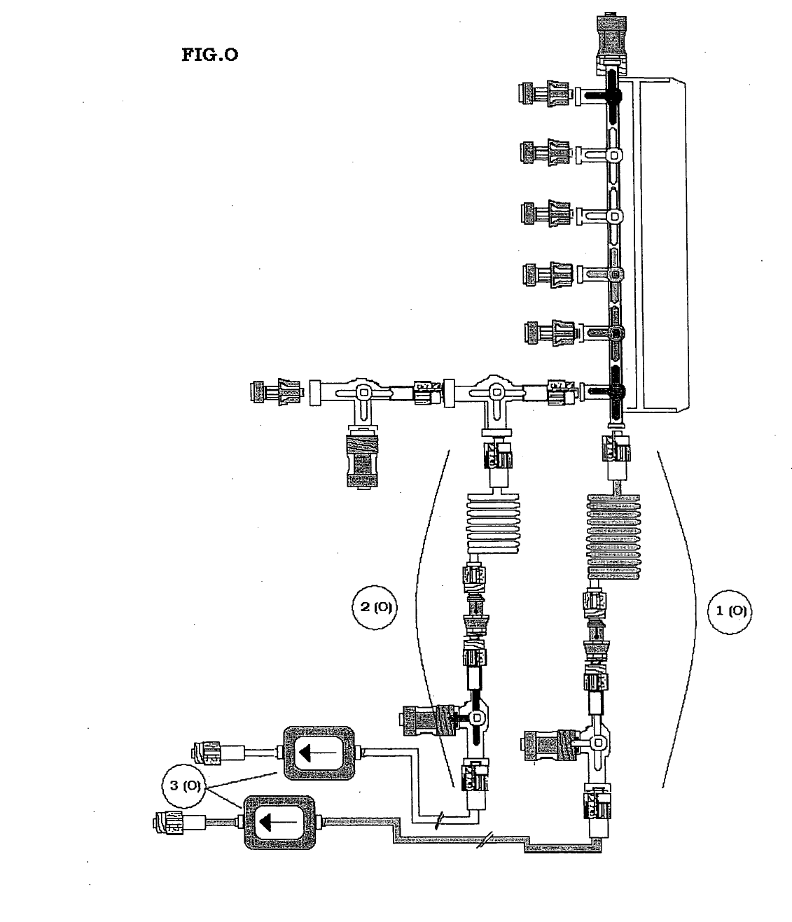

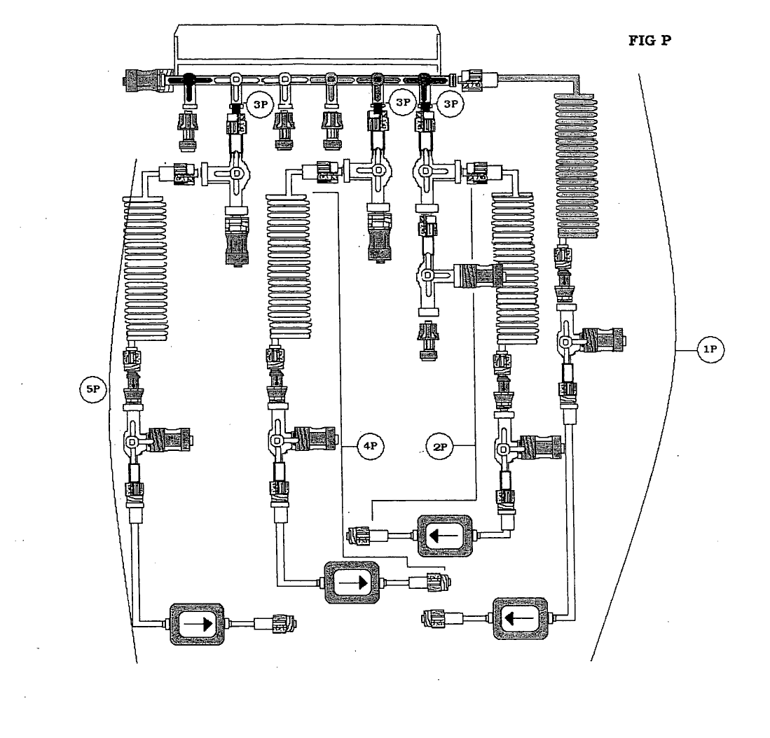

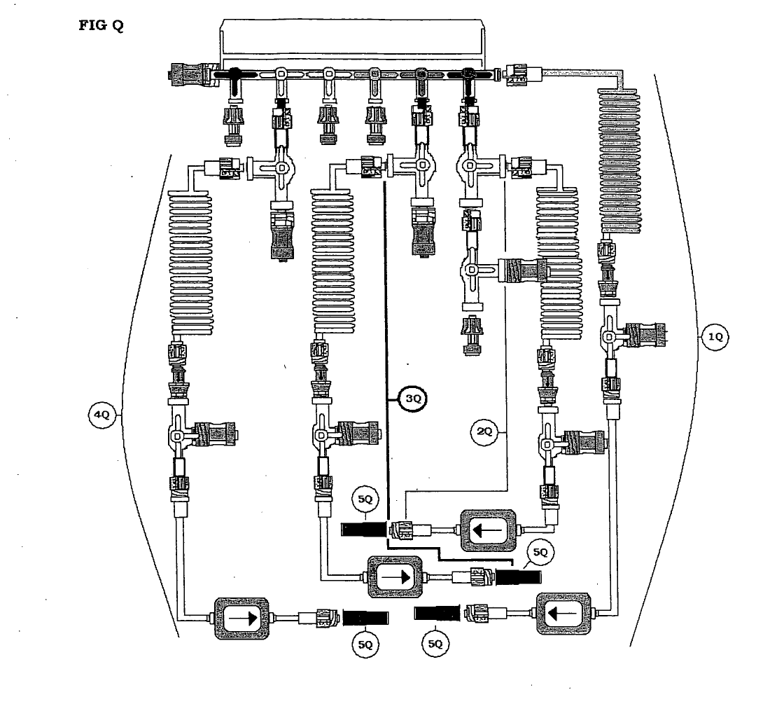

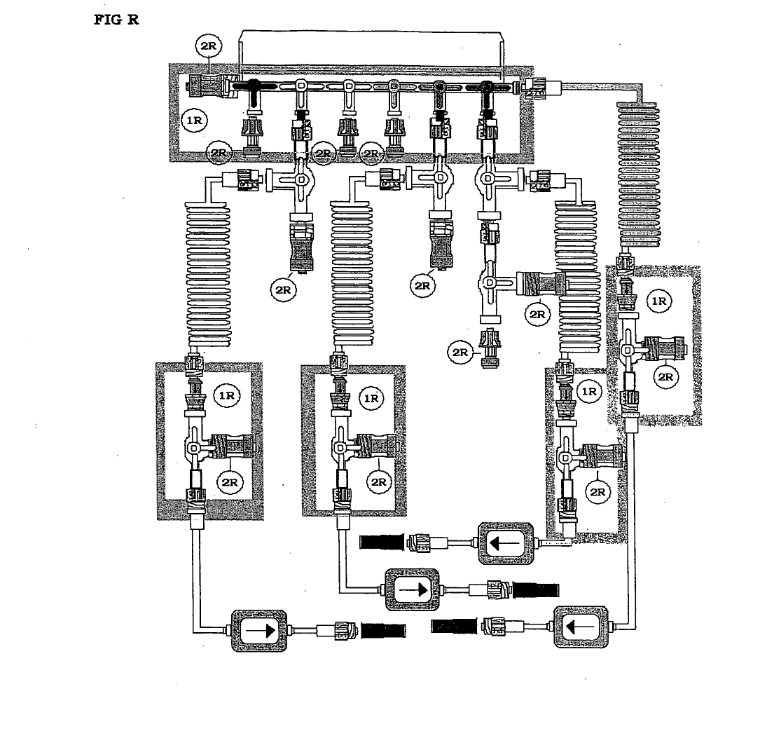

### FIELD OF APPLICATION

The present invention refers to an apparatus for administering liquids into the human body. Such apparatus is used for simultaneous and independent administration of liquids incompatible with each other.

### PRIOR ART

Apparatus for administering liquids into the human body currently available in the market (FIG. A) are made up of forced infusion systems (1A) (pumps), gravity systems (2A) (bottles/bags, connected to infusion set/extensions (3A) connected to points of access to the central venous catheter (4A).

In order to increase the possibility of having more infusion/drawing points, one or more taps (1B) or ramps (2B) directly connected to the access points of the central venous catheter (3B) are used (FIG. B); in turn pumps (5B) or bags/bottles (6B) are connected to these taps (1B) or ramps (2B) through infusion set/extensions (4B).

In order to be arranged far from the patient, ramps (1C) or taps (2C) connected to infusion set/extensions (3C) directly connected to the access points of the central venous catheter (4C) are used (FIG. C); in turn pumps (6C) or bags/bottles (7C) are connected to these taps (2C) or ramps (1C) through infusion set/extensions (5C).

The known apparatus described in figures A, B and C have the following drawbacks:
mounting one or more lines/taps/ramps several times within twelve hours, for all patients, implies wasting a lot of time; possibility of crossed contaminations and/or infections when mounting the various components of the lines given that most times the washing procedure and asepsis of the hands before wearing sterilised gloves is not followed
many supports (taps-infusion set/extensions-etc) used for simple set up of the infusion lines can be used only along with a syringe with a needle, thus increasing risks of inadvertent pricking manual assembly almost always implies a poor assembly with the risk of possible leakage of liquids from the connections
quick infusion of liquids and blood drawings performed using taps and/or ramps directly connected to the central venous catheter access systems lead to an excessive increase of hospital infections
continuous manipulation of the central venous catheter access points can cause the rupture of the same thus requiring the replacement of the central venous catheter.
risks related to air embolism given that safety systems for protecting the patient allowing discharging the air from the circuit are not used
at each infusion, nurses are forced to use an infusion set/extension to be connected to the patient; thus for example ten infusions, ten infusion set/extensions, hence leading to a tangle of pipes around the patient, implying the risk that the stitches holding the catheter might tear. In addition, the pipes could be bent occluding the passage of the liquids thus setting off the alarm of the pumps being used
use of a non-closed circuit implies the possibility of contamination and infection around and within the ramps and taps hence consequently the contamination of the infusion set/extensions and thus the patient himself.

### SUMMARY OF THE INVENTION

An object of the present invention is an apparatus for administering liquids into the human body, such apparatus comprising a main liquid loading channel and at least one secondary liquid loading channel, each channel being provided with at least one point for the loading of said liquid connectable to a respective container, and wherein the abovementioned loading channels are separated from each other using suitable means, for example membranes, able to prevent the fluids held therein from mixing.

Such object allows simultaneous administration—through the main channel and the secondary channel, connected to the central venous catheter—of various types of liquids, even incompatible with each other, without operating on the connections of the central venous catheter thus without creating problems to the patient. In addition, such object allows considerable reduction of the risks related to infection contaminations, reduction of risks related to inadvertent pricking of the operators in that the apparatus is of closed circuit type and provided with connections not requiring needles.

### BRIEF DESCRIPTION OF THE DRAWINGS

In such drawings:

FIGS. A-B-C show known systems, currently used for administering liquids;

FIG. A shows the simplest system where the pumps (1A) or bags/bottles (2A), combined with infusion set/extensions (3A), are connected directly to the central venous catheter access systems (4A);

FIG. B shows the system used to increase the points of connection of the central venous catheter (3B) where ramps (2B) and taps 1B) are interposed to the pumps (5B) and to the bags/bottles (6b) still combined with the infusion set/extensions (4B);

FIG. C shows the most complex system, but assembled on the spot, where the ramps (1C) or taps (2C) are assembled far from the points of connection (4C) of the central venous catheter through infusion set/extensions (3C); then the pumps (6C) or bags/bottles (7C) are assembled onto the ramps (1C) or taps (2C) through infusion set/extensions (5C);

FIGS. H-I-L-M-N-O-P-Q-R show the invention and its development;

FIG. H shows the initial concept of the invention wherein the two loading lines (1H), (3H) separated from each other are highlighted, such lines then being connected to the central venous catheter;

FIG. I shows the invention further provided with quick connection systems without needles (2I);

FIG. L shows the invention further provided with a plurality of points of loading (2L);

FIG. M shows the invention further provided with quick connections (4M) and unidirectional valves (3M);

FIG. N shows the invention further provided with taps (1N) and quick connection systems (2N) able for the extemporaneous infusion/drawing of liquids;

FIG. O shows the invention further provided with automatic air discharge systems (3O);

FIG. P shows the invention provided with further channels for secondary loading channels (4P) (5P);

FIG. Q shows the invention further provided with hydrophobic membranes (5Q);

FIG. R shows the invention further provided with windowed covers (1R).

### DETAILED DESCRIPTION

FIG. H shows the two loading channels (1H) (3H) joined to each other but separated by the membrane (4H), with the points of loading (2H) (5H) able to connect pumps or liquid containers. The points of loading are areas in which the liquid, coming from a suitable external container, is introduced into the circuit. Regarding the invention, the term liquid is used to indicate any substance in the liquid physical state: included in the definition of liquid substances are the pure state solutions, suspensions, dispersions, emulsions, microemulsions, colloidal systems, etc, which have such characteristics to be suitable to be injected into a patient. At their ends, the loading channels are connectable to the venous catheter and then to the patient.

FIG. I shows the two loading channels joined to each other but separated by the membrane, with the quick connection systems (2I), joined in an integral and permanent manner to the points of loading (1I) able to connect pumps or liquid containers.

FIG. L shows two loading channels joined to each other but separated by the membrane, with a plurality of quick connection systems (2L), joined in an integral and permanent manner to a plurality of points of loading (1I) able to connect pumps or liquid containers.

FIG. M shows two loading channels (1M) (2M) joined to each other but separated by the membrane, with the quick connection systems (4M) joined in an integral and permanent manner to a plurality of points of loading adapted to connect pumps or liquid containers. In addition, unidirectional valves (3M), joined in an integral and permanent manner with the aim of guaranteeing a single direction of flow of the fluids, are shown at the end of the two channels (1M) (2M).

FIG. N shows two loading channels (3N) (4N) joined to each other but separated by the membrane, with the quick connection systems, joined in an integral and permanent manner to a plurality of points of loading able to connect pumps or liquid containers. In addition, the unidirectional valves with downstream connected taps (1N) provided with quick connection systems (2N) joined in an integral and permanent manner such to allow the extemporaneous intervention of infusion and/or collection of liquids are shown at the end of the two channels (3N) (4N). The main channel (3N) and the secondary channel (4N) are distinguished from each other by the colour of the pipes (5N) and by the levers of the taps and ramps (6N).

FIG. O shows the two loading channels (1O) (2O) joined to each other but separated by the membrane, with the quick connection systems joined in an integral and permanent manner to a plurality of points of loading able to connect the pumps or liquid containers. In addition, the unidirectional valves with the downstream connected taps provided with quick connection systems joined in an integral and permanent manner such to allow the extemporaneous intervention of infusion and/or collection of liquids are shown at the end of the two main channels (1O) (2O). Automatic air discharge systems (3O) possibly present in the two channels are inserted on the main and on the secondary channels.

FIG. P shows that other loading channels (4P) (5P) still separated by a membrane (3P) can be connected to the aforementioned in FIG. O.

FIG. Q shows that other loading channels, still separated by a membrane, at whose ends are inserted capsules with hydrophobic membrane (5Q) for eliminating air during the initial filling of the channel, can be connected to the aforementioned in FIG. P.

FIG. R shows that a system for protecting the connection points (2R) characterised by windowed openings (1R) is joined to the aforementioned in FIG. Q.

The present invention allows simultaneous administration of liquids incompatible with each other, using quick and safety connection systems fixed to the invention in an integral and permanent manner in order to obtain a closed circuit system capable of minimising the possibilities of contamination before and during the therapy, within a determined range of time, for example up to 96 hours, due to the peculiarity and the chemical/physical characteristics of the components used. The present invention offers the possibility to have a closed circuit system ready for use, capable of minimising the possibilities of contaminating the patient; through the quick connection systems the invention protects the operators from the risk of inadvertent pricking, allows simultaneous administration of more than one drug to the patient, minimises the tangles around the patient.

1. Apparatus for administering mutually incompatible liquids into the human body comprising a main liquid loading channel (1H) and at least one secondary liquid loading channel (3H), each channel being provided with at least one point (2H), (5H) for introducing said liquid, connectable to a respective container, wherein said loading channels are separated from each other by suitable means able to prevent the fluids held therein from mixing.

2. Apparatus according to claim 1 characterized in that the points of loading of the main and/or secondary channel are equipped in an integral manner with quick connection systems and without needles (21)

3. Apparatus according to claim 1 characterized in that the points of loading of the main and/or secondary channels comprise multiple points of loading (2L).

4. Apparatus according to claim 1 characterized in that unidirectional apparatus (3M) serving to guarantee a single direction of flow of the liquid are inserted at the ends of the main and/or secondary channels.

5. Apparatus according to claim 4 characterized in that the use of quick connections (2I), (4M) on all the points of loading of the main and/or secondary channels and the insertion of unidirectional apparatus (3M) make the apparatus a closed circuit.

6. Apparatus according to claim 1 characterized in that taps (1N) are provided with quick connection systems (2N) such to allow the extemporaneous intervention of infusion and/or drawing of liquids are inserted on the main and/or secondary channel.

7. Apparatus according to claim 1 characterized in that the main channel and the secondary channel are distinguished from each other by the colour color of the pipes (5N) and by the levers of the taps and by the ramp (6N).

8. Apparatus according to claim 1 characterized in that automatic systems (3O) for discharging the air possibly present in the channel are inserted on the main and/or secondary channel.

9. Apparatus according to claim 1 characterized in that it comprises further secondary liquid loading channels (4P) (5P).

10. Apparatus according to claim 1 characterized in that a capsule with a hydrophobic membrane (5Q) allowing the elimination of air only during the step of purshing (initial operation of filling the channels), without wasting liquids is inserted at the ends of the main and/or secondary channel.

11. Apparatus according to claim 1 characterized in that it is supplemented with windowed covers (1R) to protect the connection points (2R) from bacteria, such covers (1R) being treatable with disinfectant solutions.
